(19) United States
(12) Patent Application Publication
(10) Pub. No.: US 2016/0015619 A1
(43) Pub. Date: Jan. 21, 2016

(54) TRANSPARENT SOL, GEL COMPOSITION CONTAINING HIGH CONCENTRATIONS OF VITAMIN C AND VITAMIN C DERIVATIVE

(71) Applicant: ABIO CO., LTD., Yuseong-gu Daejeon (KR)

(72) Inventor: Tae Yeong LEE, Busan (KR)

(21) Appl. No.: 14/767,298

(22) PCT Filed: Jul. 26, 2013
(86) PCT No.: PCT/KR2013/006735

§ 371 Date: Aug. 12, 2015

(30) Foreign Application Priority Data
Feb. 12, 2013 (KR) 10-2013-0014938

(51) Int. Cl. A61K 8/67; A61K 8/34; A61K 8/04; A61Q 19/08; A61Q 19/00; A61K 8/73; A61Q 19/02

(52) U.S. Cl. CPC A61K 8/676; A61K 8/735; A61K 8/345; A61Q 19/02; A61Q 19/08; A61Q 19/007; A61K 8/34; A61K 8/042

(57) ABSTRACT

The present invention relates to a transparent sol, gel composition containing high concentrations of vitamin C and vitamin C derivative. More specifically, disclosed is a technical field associated with a vitamin C composition, which is usable as an essence, a mask pack, a hydrogel mask, or the like, by making a composition containing a high concentration of vitamin C or vitamin C derivative, a polyalcohol, an acid mucopolysaccharide, and a hydrophilic solvent, into a sol or gel so as to stabilize the high concentration of vitamin C or vitamin C derivative in an aqueous solution. In addition, the present invention has a dosage form of a high-viscosity sol or gel phase since the vitamin C or the vitamin C derivative is dissolved in the aqueous solution, and therefore, provided is a high-viscosity composition having effects of suppressing carbon dioxide which may be generated when vitamin C is unstable, not easily deteriorating by minimizing the contact with air due to little movement when being carried or stored, being convenient to use, having external beauty due to a transparent appearance, and being stable.

### TECHNICAL FIELD

The present invention relates to a transparent sol, gel composition containing high concentrations of vitamin C and vitamin C derivative, and in particular to a vitamin C composition which can be used to manufacture an essence, a mask pack or a hydrogel mask in such a way to make vitamin C of a high concentration or vitamin C derivative in an aqueous liquid phase into a stable, high viscosity sol from or a stable high viscosity gel form and to make, into a sol or a gel, a composition containing vitamin C of a high concentration or vitamin C derivative of a high concentration, a polyhydric alcohol and an acidic mucopolysaccharide and a hydrophilic solvent.

### BACKGROUND ART

Vitamin C is one of trace elements which allow to keep the function and healthy state of a human body and is called an L-ascorbic acid. It allows a human body to resist any infection and heal injuries and keep tissues healthy and is one of antioxidants which prevent any damage to cells due to free radical. Vitamin C is contained in fruits, vegetables, etc. and is rich in persimmon, orange, tomato, broccoli, spinach, strawberry, melon, potato, etc.

In addition, vitamin C is an assistant factor which plays a key role in generating collagen while increasing an elasticity of skin, preventing any small wrinkles, promoting a recovery of cuts and helping a recovery after laser treatment or surgical operation.

In addition, vitamin C allows to enhance reproduction after a treatment of skin or erythema skin, which is underway of an expansion of capillary the function of which is weak or acne or after a skin peeling operation. Namely, vitamin C can provide a whitening effect by inhibiting tyrosinase which forms a melanin pigment and an anti-oxidation operation during which melanin is reduced, while preventing a pigmentation of melasma, freckle, etc. and thinning such pigmentations. Vitamin C also allows to inhibit aging in such a way to eliminate harmful oxygen generating due to various incomplete metabolism in a human body, ultraviolet rays, etc. and enhance a resistance force of a skin and make it healthy.

Since such vitamin C has a close relationship with the skin, cosmetic containing vitamin C is being manufactured or sold. The vitamin C is stable in a crystal dry state, but has a stability wherein it is fast oxidized by moisture, air, light, heat and is destroyed, so there exist a lot of difficulties. For this reason, various vitamin C derivatives are being developed or are under development. In case of a conventional vitamin C research and development, stabilization has been a key issue during the research. There have been a lot of difficulties in thickening vitamin C due to a low PH value. To this end, a new technology is needed, which makes it possible to make a conventional vitamin C or vitamin C derivative into a stable solution which is easy to use in a method for manufacturing a sol or gel of a high viscosity using vitamin C or a stabilized vitamin C derivative.

As a technology for stabilizing vitamin C related to the above method, there is the Korean patent publication number 10-2003-0048725 which is directed to a cosmetic composition which is dispersed in one or two or more kinds of skin friendly solutions and is contained in a silicon capsule containing a predetermined size of the dispersed liquid wherein a prepared capsule is dispersed in a secondary skin friendly silicon oil or a natural oil or a composite solvent.

The Korean patent publication number 10-2005-0019668 discloses an anhydrous oily cosmetic composition containing vitamin C wherein an unstable pure vitamin C is absorbed into silicon powder using a polar solvent excluding an unstable pure vitamin C, and the silicon powder which has absorbed the pure vitamin C is prepared into an oily formulation using silicon oil, thus minimizing any skin stimulus by increasing an oxidation stability of vitamin C and effectively improving whitening and skin elasticity.

The Korean patent publication number 10-2004-0085825 discloses a method for largely inhibiting the decomposition of vitamin C in such a way to stabilize the vitamin C which is melted in an aqueous solution using CMC (Carboxymethlycellulose).

However, the above listed patent publications may generate sediment due to the generated complex of the CMC and the vitamin C. In order to prevent such a complex, the pH range is adjusted to a neutral range, so the characteristic of the stable vitamin C in a low pH range may lose, thus allowing the CMC to form a complex, whereupon it is impossible to expect any thickening effect. The forms of a sol or a gel may be obtained only by using over amount of the CMC, which causes a bad use feeling, entailing uneasiness when in use. Due to an anhydrous solution, a dissolution of high concentration vitamin C is hard, and precipitation and sediment may occur due to a non-dissolved vitamin C, and there may be any stimulus from vitamin C.

In addition, the Korean patent publication number 10-2007-0079166 discloses a whitening cosmetic composition which improves any inconvenience when in use, for example, sticky feeling, pushing back, etc. due to the vitamin C derivative when a silicon emollient while preventing the decomposition due to an external environment, for example, moisture, temperature and air, by collecting a large content of vitamin C or a vitamin C derivative in the inner phase of the formulations of aqueous phase in oil and stabilizing them. However, the above mentioned problems may occur because of the formulation of cream phase due to the uses of anhydrous solution and silicon emollients.

In addition, the Korean patent registration number 10-051691 discloses a composition which stabilizes vitamin C in an aqueous phase and a method for an encapsulation based on a complex reaction of a cation substance and an anion substance (vitamin C) while stabilizing vitamin C using the same, wherein vitamin C or its derivative is dissolved in water and becomes anion which reacts with cation polymer, thus forming a stable acid-base complex, whereupon vitamin C and its derivative are first stabilized, and its complex is secondarily encapsulated by anion polymer, thus more stabilizing vitamin C and its derivative.

However, since the acid-base complex of vitamin may form precipitation due to a difference in solubility with respect to solvent, it needs to adjust pH, if necessary, to obtain a stable reaction and a product of acid and base complex. An inducement for an accurate reaction is necessary. A secondary reaction and complex may occur due to multiple reaction additives, and since vitamin C is added with cation and anion being coexisting, precipitation or a precipitation of complex salt may occur due to change over time.

The cosmetic made from vitamin C or its derivative prepared by the conventional method has succeeded a little in stabilizing vitamin C, but it exists in a liquefied form and has a low viscosity, so it may flow down when in use, and in the formulation of a mask pack, since viscosity is not high, it is hard to use, and the formulation of a hydrogel type is impossible.

### DISCLOSURE OF INVENTION

### Technical Problem

The present invention is made in an effort to improve the above-described problems. An aqueous solution of vitamin C is acidic and has a high acidic value at a high concentration, so a thickener which is generally used causes a clot, for example, a complex formation, etc. to precipitate or even though it is thickened, its effect may not be good, which means that a lot of amount is necessary when in use. The use of a lot of amount of thickener may cause a problem wherein the cosmetic may feel sticky during use or a tightening feeling may occur after use.

In addition, since vitamin C is made into liposome or is encapsulated and is added into the formulation of cosmetic and is emulsified and then is used, it is an opaque formulation, which means that it is not a transparent liquefied formulation of vitamin C or its derivative, so it is impossible to visually provide a vitamin C effect, and there is a limit preparing a high content of vitamin C formulation.

Since it is anhydrous, viscosity may be thus adjusted by dispersing in oily one or silicon type oil, however such a mixture may not be seemed like a vitamin C solution or its derivative solution, and a particle of the dispersed vitamin C or its derivative may stimulate a skin. Accordingly, it is an object of the present invention to provide a solution with respect to the above-described problems.

### Solution to Problem

To achieve the above object, there is provided a transparent sol and gel composition containing high concentrations of vitamin C and vitamin C derivative, without using a conventional thickener, which may include, but is not limited to, 0.2˜20% by weight of vitamin C or vitamin C derivative; 20˜80% by weight of polyhydric alcohol; 0.05˜3% by weight of acidic mucopolysaccharide; and 15˜60% by weight of a hydrophilic solvent.

### Advantageous Effects

The transparent sol and gel composition containing high concentrations of vitamin C or vitamin C derivative according to the present invention has an effect on inhibiting carbon dioxide which may occur if vitamin C is unstable, since it has a sol or gel formulation of a high viscosity in terms of its form in a state where vitamin C or vitamin C derivative is dissolved in an aqueous solution and has another effect on that it does not easily go spoiled by minimizing any contact with air since there is little movement when storing by carrying with. It is possible to obtain a stable, high viscosity composition since the use is easy, and a vitamin C solution looks beautiful thanks to a transparent exterior.

### BRIEF DESCRIPTION OF DRAWINGS

FIG. 1 is a graph illustrating the viscosity of the experimental examples 1˜4 according to the present invention.

FIG. 2 is a photo illustrating a discoloration for 1˜4 weeks at 45° C. of exemplary embodiments 1˜5 of the table 5 and comparative examples 1˜2.

FIG. 3 is a transparent gel photo prepared according to the exemplary embodiments of the present invention.

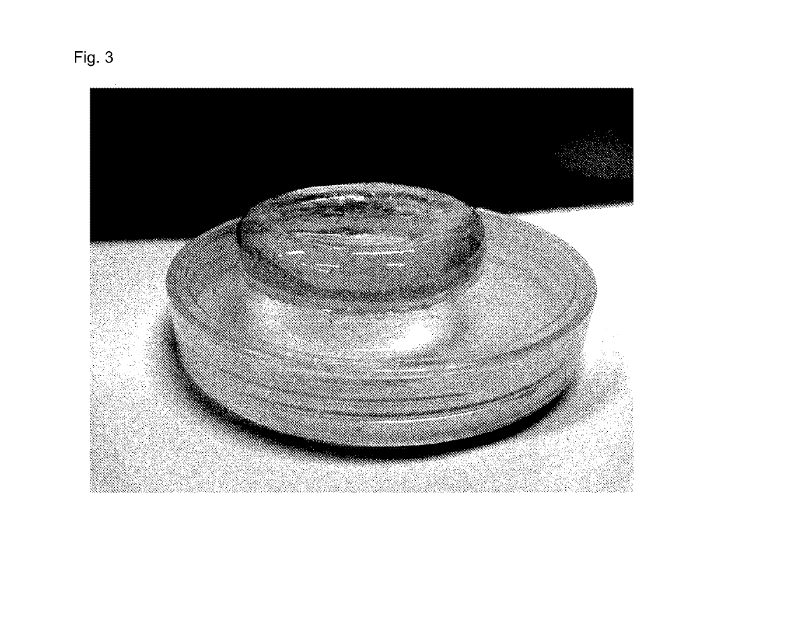

### BEST MODES FOR CARRYING OUT THE INVENTION

The present invention is directed to a sol or gel composition wherein its aqueous phase is acidic and shows a high acidic value at a high concentration, thus thickening, without using a conventional thickener, of vitamin C or vitamin C derivative having high concentration the viscosity of which is hard to increase using a conventional thickener.

More specifically, vitamin C or its vitamin C derivative is made into a gel form by inputting powder or solution of vitamin C or vitamin C derivative of high concentration into prepared hydrophilic solution, thus preparing a sol or gel of vitamin C or vitamin C derivative. Since the prepared sol or gel does not flow down, its use is convenient, and since motion is less while it is being moved, which means minimizing any contact with air, it does not easily go spoiled. The present invention allows to achieve a stabilization of unstable vitamin C or vitamin C derivative because it has a high viscosity. The thusly prepared sol or gel of vitamin C or vitamin C derivative of high concentration may be used to manufacture an essence, a mask pack or a hydrogel mask.

The composition of the present invention may include, but is not limited to, 0.2˜20% by weight of vitamin C or vitamin C derivative; 20˜80% by weight of polyhydric alcohol; 0.05˜3% by weight of acidic mucopolysaccharide; and 15˜60% by weight of a hydrophilic solvent.

More specifically, during a gelation reaction, a sole or gelation is performed since thickening is performed by inputting powder or solution of vitamin C or vitamin C derivative into a polyhydric alcohol and an acidic mucopolysaccharide solution and a hydrophilic solvent without using any thickener. At this time, the acidic mucopolysaccharide may be used in a form of powder or solution, but for the sake of a complete dissolution, a previously melted solution is appropriate, and if necessary, a hydrophilic solvent may be used so as to adjust viscosity before or after gelation reaction.

Such a gelation reaction occurs only when there exist a polyhydric alcohol and an acidic mucopolysaccharide solution and a vitamin C or vitamin C derivative solution. In an exemplary embodiment, even if a solution of vitamin C or vitamin C derivative is inputted in glycol, a gelation reaction does not occur, and even if vitamin C or its derivative is mixed in a hyaluronic acid natrium solution, a gelation reaction does not occur. In the same way, a gelation reaction does not occur only with a polyhydric alcohol and a hyaluronic acid natrium solution.

Namely, as a result of a gelation reaction, the viscosity of solution increases after mixing than the highest level of the viscosities of each solution, and at the highest viscosity, it becomes a sticky and dense liquid sol state at the highest viscosity or it loses liquidity and becomes a gel state. The thusly prepared sol or gel may be used to manufacture a high viscosity essence or a hydrogel or a gel mask. In addition, it is obvious for a person having ordinary skill in the art to add a perfume, an antiseptic, a metallic ion masking agent and other additives, if necessary.

In addition, a pure vitamin C is stable in a crystal dry state, but it has an instability wherein it is fast oxidized by moisture, air, light or heat and is destroyed. So, it can be inputted into a human body in a stable state using a vitamin C derivative, and then the derivative is removed, and the vitamin C derivative becomes vitamin C, which allows to keep the functions of a human body and the human body healthy.

Therefore, the sol and gel of vitamin C or vitamin C derivative containing high concentration are easy to use and allow to increase skin flexibility like a conventional vitamin C solution and prevent small wrinkles and promote a recovery of cuts and provide a fast recovery after laser treatment or surgical operation.

In addition, the vitamin C or vitamin C derivative containing high concentration consists of any of L-ascorbic acid and sodium ascorbylphosphate (SAP), magnesium ascorbylphosphate (MAP), calcium ascorbylphosphate, ascorbic acid polpeptide, ascorbyl dipalmitate, ascorbyl palmitate, ascorbyl glucoside, ethyl ascorbyl ether, ascorbyl stearate and ascorbyl ethylsilanolpectinate, and it is preferred that 0.2˜20% by weight is contained as a composition ratio. If it is contained less than 0.2% by weight, a gelation does not occur well and viscosity is low, so it may flow down, which means that it is not easy to use. If it is contained over 20% by weight, dissolubility may be low depending on temperature, thus forming sediment.

The polyhydric alcohol of the present invention means alcohol which has multiple hydroxyl groups (OH) in a generally known molecular and mixes well with water like monohydric alcohol, and it is preferred that it consists of at least one of glycol, 1,3-butylene glycol, propylene glycol, sorbitol and glycerin. Namely, the polyhydric alcohol is a component the stability of which has been proved as a substance which has been long used as a cosmetic moisturizer.

Namely, the polyhydric alcohol according to the present invention is contained at a composition ratio of 20˜80% by weight, and if the polyhydric alcohol is contained less than 20% by weight or over 80% by weight, the increase of the viscosity may slow down during a gelation reaction, whereupon it is impossible to obtain the effects of the present invention.

Next, the mucopolysaccharide collectively means polysaccharide which contains amino acid existing as a protein complex in an animal tissue and is called glycosaminoglycan. In addition, different from other saccharides (glucose, glycogen), it does not turn into a biological energy source after metabolism, and it plays a key role in maintaining of tissues and its strength and flexibility as an organizational substance of the tissues and has a function, for example, a capacity adjustment of an extracellular fluid, a movement of catalyst, and a balance and a deposition in a tissue of calcium. The above-described the mucopolysaccharide may be categorized into a neutral mucopolysaccharide which does not have any acid group and a mucopolysaccharide which has an acidic group. As an example of the former, there is chitin, and as an example of the latter, there are hyaluronic acid, chondroitin sulfuric acid, heparin, etc. It is present in a joint fluid, etc. and may be extracted from cockscombs. In recent years, it is possible to manufacture at low cost using microorganism.

The acidic mucopolysaccharide, for example, hyaluronic acid, chondroitin sulfuric acid, etc. which exist in a skin have a high water holding capacity as a protein complex in a connective tissue and allows for a smooth metabolism by providing an appropriate moisturizing environment to cells, by means of which a cell maintenance function can be obtained, thus providing lubrication and flexibility to a skin, and the skin can be protected against penetrations of external force, bacteria, etc.

In addition, hyaluronic acid and chondroitin sulfuric acid are developed as a moisturizing agent and is currently being used as a feeling improver which provides a sleek feeling to the product. Such a moisturizing agent has a feature in that it has a high moisturizing capacity. A good moisturizing effect can be provided by using mucopolysaccharide in a single form or by combining two kinds of mucopolysaccharides in the way that the above moisturizing agent forms a substrate containing a lot of moisture in an engaged state with respect to collagen fiber and mucopolysaccharide in a thick skin.

Thanks to the above features, the present invention consists of any of hyaluronic acid natrium, chondroitin sulfuric acid and heparin among acidic mucopolysaccharides at a composition ratio of 0.05˜3% by weight. Namely, the mucopolysaccharide which is a key component of the gelation reactant used in the present invention, is recognized as having a stability in the cosmetic business and plays a key role in the generation of collagen which is related to the aging of a skin, which means that there is not limit in use.

At this time, the acidic mucopolysaccharide may be used in a form of powder or solution as described above, but a solution type is preferred, wherein it is previously melted for a complete dissolution. If the acidic mucopolysaccharide is less than 0.05% by weight, the increase of the viscosity of the composition of the present invention may too slow down, which means that it is impossible to obtain effects of the present invention, and if it is used over 3% by weight, oligomer hyaluronic acid the molecular weight of which is relatively less among hyaluronic acids may not be completely dissolved, thus forming sediment.

For this, it is preferred that the present invention uses hyaluronic acid among acidic mucopolysaccharides since the hyaluronic acid is a polymer compound and may be categorized by molecular weight into HMW, MMW, LMW, VLMW and oligo HA (oligomer hyaluronic acid). If the hyaluronic acid with a high molecular weight is used, the effect on increasing viscosity is high, but due to a problem in cost and dissolubility, it is preferred to completely dissolve hyaluronic acid natrium of 0.5˜1.8 Mda into 1% of hyaluronic acid natrium aqueous solution.

Next, hydrophilic solvent of the present invention is used to adjust viscosity and consists of any of distilled water and ethyl alcohol and may further include distilled water which is a solvent used to dissolve mucopolysaccharide, and the entire portions are included at a composition ratio of 15˜60% by weight because if the hydrophilic solvent is less than 15% by weight, it is hard to melt vitamin C and acidic mucopolysaccharide, thus forming sediment, and if it is used over 60% by weight, viscosity lowers, which makes hard a gelation reaction. In addition, if ethyl alcohol is mixed and used, ethyl alcohol uses 0˜20% by weight of the entire hydrophilic solvent because if the ethyl alcohol is used over 20% by weight of the entire hydrophilic solvent, a skin may has a stimulation, so an uneasy feeling may occur when using the composition of the present invention.

The present invention will be described in details with reference to the experimental examples and exemplary embodiments below, but it is not limited thereto.

The experiments were performed with the following compositions so as to show a gelation reaction of the present invention, and the viscosities were measured. At this time, the viscosity measuring device used was Brookfield Digital Viscometer LVDV-I Prime viscometer, and the spindle No. 61˜64 was used depending on viscosities, and the measurements were performed at RPM 50.

### Experimental Example 1

Table 1 shows a result of the viscosity measurements performed by mixing 1,3-butylene glycol as polyhydric alcohol and 1% of hyaluronic acid natrium solution.

TABLE 1

| Component (% by weight) | 1 | 2 | 3 | 4 | 5 | 6 |
|---|---|---|---|---|---|---|
| 1.3-BG | 100 | 80 | 60 | 40 | 20 | 0 |
| 1% Na-hyaluronic acid | 0 | 20 | 40 | 60 | 80 | 100 |
| Viscosity (c.p.s) | 187.2 | 1435 | 1627 | 1908 | 1109 | 1073 |

### Experimental Example 2

The viscosities were measured by adding 10% by weight of distilled water of the composition of the experimental example 1, and a result of the measurements is shown in Table 2.

TABLE 2

| Component (% by weight) | 1 | 2 | 3 | 4 | 5 | 6 |
|---|---|---|---|---|---|---|
| 1.3-BG | 90 | 72 | 54 | 36 | 18 | 0 |
| 1% Na-hyaluronic acid | 0 | 18 | 36 | 54 | 72 | 90 |
| DW | 10 | 10 | 10 | 10 | 10 | 10 |
| Viscosity (c.p.s) | 67.2 | 853.4 | 1056 | 1444 | 897.4 | 574.3 |

### Experimental Example 3

10% by weight of 20% vitamin C aqueous solution was added to the composition of the experimental example 1. At this time, the viscosity of 20% vitamin C aqueous solution was 46 C.P.S.

TABLE 3

| Component (% by weight) | 1 | 2 | 3 | 4 | 5 | 6 |
|---|---|---|---|---|---|---|
| 1.3-BG | 90 | 72 | 54 | 36 | 18 | 0 |
| 1% Na-hyaluronic acid | 0 | 18 | 36 | 54 | 72 | 90 |
| 20% vitamin C aqueous | 10 | 10 | 10 | 10 | 10 | 10 |
| solution | | | | | | |
| Viscosity (c.p.s) | 114.6 | 3530 | 6513 | 3779 | 2347 | 890.2 |

### Experimental Example 4

In the composition of the experimental example 3, glycerin was used instead of 1,3-butylene glycol, and sodium ascorbyl phosphate, which is vitamin C derivative, was used instead of 20% vitamin C aqueous solution.

TABLE 4

| Component (% by weight) | 1 | 2 | 3 | 4 |
|---|---|---|---|---|
| Glycerin | 72 | 54 | 36 | 18 |
| 1% Na-hyaluronic acid | 18 | 36 | 54 | 72 |
| 20 A.S.P | 10 | 10 | 10 | 10 |
| Viscosity (c.p.s) | 5713 | 4907 | 3124 | 1986 |

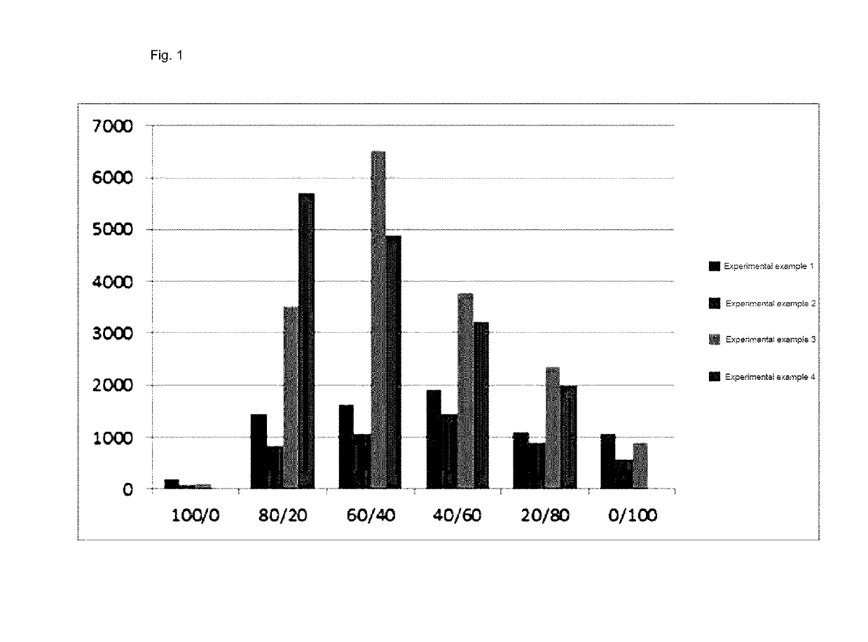

As confirmed through the above experiments, viscosity was increased slightly by mixing polyhydric alcohol and hyaluronic acid natrium, and viscosity was decreased by adding distilled water to the solution mixed with polyhydric alcohol and hyaluronic acid natrium. It was confirmed that the viscosity was sharply increased by mixing a vitamin C aqueous solution with a very low viscosity with the solution mixed with polyhydric alcohol and hyaluronic acid natrium. In addition, as confirmed in the experimental example 4, the same effects were obtained when using glycerin and vitamin C derivative. This is shown in the graphic in FIG. 1.

As seen in Table 5, the samples were prepared according to the embodiments 1˜5 and the comparative examples 1˜2, and discoloration, anti-oxidation effects, stability evaluation of vitamin C, whitening effects, and skin moisturizing effects were tested and compared.

TABLE 5

| Components | Embodiment | Embodiment | Embodiment | Embodiment | Embodiment | Comparative | Comparative |
|---|---|---|---|---|---|---|---|
| (weight) | 1 | 2 | 3 | 4 | 5 | example 1 | example 2 |
| 1.3-BG | 68 | 51 | 51 | 34 | 34 | — | — |
| 1% Na- | 17 | 34 | 34 | 51 | 51 | — | — |
| hyaluronic | | | | | | | |
| acid | | | | | | | |
| EDTA-2NA | 0.02 | 0.02 | 0.02 | 0.02 | 0.02 | 0.02 | 0.02 |
| L-ascorbic | 10 | 10 | — | 10 | — | 10 | — |
| acid | | | | | | | |
| Ascorbyl | — | — | 10 | — | 10 | — | 10 |
| glucoside | | | | | | | |
| (DW/alcohol) | to 100 | to 100 | to 100 | to 100 | to 100 | to 100 | to 100 |
| 1:1 solution | | | | | | | |

### Experimental Example 5

### Discoloration Experiment

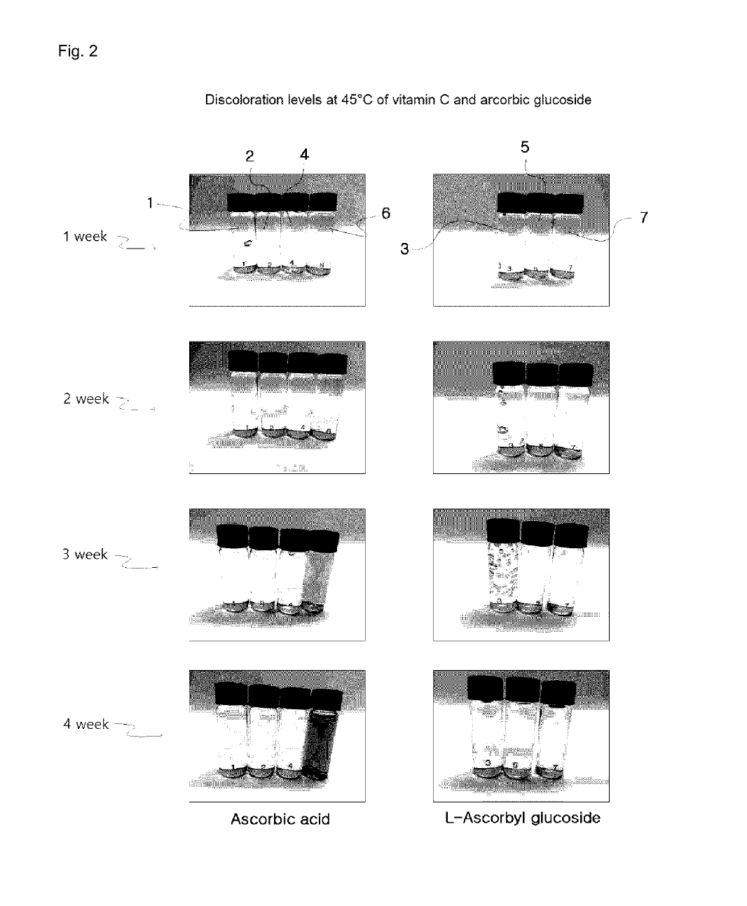

The cosmetic compositions prepared by the embodiments 1˜5 and comparative examples 1˜2 in Table 5 were stored for 4 weeks in an opaque glass container in a constant temperature oven where the temperature was constantly maintained at 45° C., and discoloration and smell change were measured. A result of the measurement is shown in Table 6. At this time, the degrees of discoloration and smell change of products were categorized into the following 6 levels, and the photos of actual experiments are attached to FIG. 2.

TABLE 6

| | Embodiment | Embodiment | Embodiment | Embodiment | Embodiment | Comparative | Comparative |
|---|---|---|---|---|---|---|---|
| | 1 | 2 | 3 | 4 | 5 | example 1 | example 2 |
| levels | 2 | 2 | 1 | 3 | 2 | 6 | 4 |

A comparison was performed in such a way that the embodiments 1, 2 and 4 and the comparative example 1 used a pure L-ascorvic acid, and the embodiments 3 and 5 and the comparative example 2 used ascorvyl glucoside which is a stable derivative of vitamin C. As a result, the embodiments 1, 2 and 4 were better in terms of a discoloration stability as compared to the comparative example 1, and the embodiments 3 and 5 were better in terms of a discoloration stability as compared to the comparative example 2, so can be seen that example 1, 2, 3, 4 and 5 more stable. Namely, it was possible to confirm that the vitamin C was stabilized if it had a sol or gel shape of a high viscosity in vitamin C solution of a high concentration.

### Experimental Example 6

### Anti-Oxidation Experiment

The free radical removing capability was experimented using the embodiment 4 in Table 5. In case of the DPPH (1,1-diphenyl-2-picrylhydrazyl) used, the radical removing capability of radical sample which changes colors depending on the changes in oxidation/reduction environment was confirmed through the measurements of the O.D. value.

In details, the DPPH experiment sample was prepared by inputting the solution of the embodiment 4 in Table 5 into each well of the 96-well plate by 100 μl. The DPPH was added by 10 μl, and the mixture was placed at a room temperature for 30 minutes, and the absorbance at 517 nm was measured using a micro plate reader. At this time, the solution prepared by adding 10% of the distilled water instead of vitamin C into the solution of the embodiment 4 and the pure vitamin C were used as a control group. A result is shown in Table 7.

TABLE 7

| | Activation (%) of anti-oxidation |
|---|---|
| Embodiment 4 | 91.6 |
| Control group 1 (solvent) | 2.8 |
| Control group 2 (vitamin C) | 90.8 |

As seen in Table 7, it was recognized that the composition of the hydrophilic solvent of the control group 1 had a minute effect on the activation of the anti-oxidation, and the composition of the pure vitamin C of the control group 2 had a high effect on the activation of the anti-oxidation which was known in the art, and it was possible to confirm that the composition of the embodiment 4 of the present invention had a similar effect on the activation of the anti-oxidation of the pure vitamin C composition of the control group 2.

### Experimental Example 7

### Stability Evaluation

In order to test the stability of the composition of the present invention, the embodiments 1˜5 and comparative examples 1˜2 were stored for 4 weeks in an opaque glass container in a constant temperature oven where the temperature was constantly maintained a room temperature and at 45° C., and the amounts of the remaining vitamin C and Vitamin C derivative were relatively determined using the peat area which showed 266 nm using a high performance liquid chromatography (HPLC) and were calculated based on the following formula and were shown in Table 8.

$$Titer ( \% ) = \frac{Amount of remaining vitamin C after 4 weeks}{Amount of initial vitamin C} \times 100$$

TABLE 8

| Classification | Titer (%) of vitamin C | Experiment conditions |
|---|---|---|
| Embodiment 1 | 93 | 45° C. |
| Embodiment 2 | 91 | |
| Embodiment 3 | 95 | |
| Embodiment 4 | 76 | |
| Embodiment 5 | 88 | |
| Comparative example 1 | 11 | |
| Comparative example 2 | 71 | |

As seen in Table 8, in the embodiments 1, 2 and 4 and the comparative example 1 the experiments on which were performed using the pure L-ascorbic acid, it was recognized that the amount of the vitamin C remaining in the compositions of the embodiments 1, 2 and 4 was greater than the composition of the comparative example 1, and in the embodiments 3 and 5 and the comparative example 2 which used an ascorbyl glutamate which was a stable derivative of vitamin C, it was recognized that the amount of the remaining vitamin C of the embodiments 3 and 5 was greater than the composition of the comparative example 2. Namely, it was known that the embodiments 1˜5 of the present invention had higher stability than the comparative examples 1 and 2.

### Experimental Example 8

### Experiments on Skin Whitening Effects

In order to perform an experiment on the skin whitening effects of the present invention, an opaque tape with 7 holes each having a diameter of 1.5 cm was attached onto a portion of an upper forearm of a testee with respect to 14 healthy women over 20 years, and UVA which was 1.5˜2.0 times the minimal erythema dose of each testee was irradiated in order to induce a blackening of a skin. Next, the essence made from the compositions of the embodiments 1˜5 and the comparative examples 1˜2 was coated twice per day (morning, afternoon), and the light and shade of the skin were measured after two months using the chroma meter.

The effects were determined by obtaining an “L” value which meant the light and shade of the skin (The skin color of Korean who was not burnt had values of 50˜70). If there were not any effects after the test substance was coated the value L gradually increased, and the comparison between the test substances was expressed as a value of ΔL (the final value L−the value L before the coating of essence). Higher value of ΔL meant a higher whitening effect. The values of ΔL based on a result of experiments were shown in Table 9, and the average values of the value L were recorded.

TABLE 9

| | Value L | | Whitening |
|---|---|---|---|
| | before coating | Final value L | effects (ΔL) |
| Embodiment 1 | 43.37 | 46.42 | 3.05 |
| Embodiment 2 | 43.37 | 46.50 | 3.13 |
| Embodiment 3 | 43.37 | 46.94 | 3.57 |
| Embodiment 4 | 43.37 | 46.55 | 3.18 |
| Embodiment 5 | 43.37 | 46.86 | 3.49 |
| Comparative example 1 | 43.37 | 46.38 | 3.01 |
| Comparative example 2 | 43.37 | 46.48 | 3.11 |

As seen in Table 9, it was possible to recognize that the compositions of the embodiments 1˜5 of the present invention had whitening effects with the aid of vitamin C like in the comparative examples 1˜2.

### Experimental Example 9

### Experiments on Skin Moisturizing Improvement Effects

For the experiments of the skin moisturizing improvement effects of the present invention, the evaluation was performed with respect to 30 healthy women which were over 20 years. 20 μl of the essence made from the composition of the embodiment 4 and the comparative example 1 was coated.

The average value was calculated by measuring thrice in a row six hours after the coating.

The moisturizing capacity measuring device was the MSA pro of Tru system (Korea), and the experiments were performed in the constant temperature and humidity room of the relative humidity of 50% at 25° C. Higher values meant higher moisturizing effects.

The method for calculating moisturizing capacity is as follows.

Moisturizing capacity(%)=[1+(Mt−Mo)]/Mo×100

Mt: the value of moisturizing capacity after time t

Mo: the value of measurement before coating

TABLE 10

| Moisturizing capacity (%) | Before coating | 6 hours after coating |
|---|---|---|
| Embodiment 4 | 100 | 128.91 |
| Comparative example 1 | 100 | 87.06 |

As seen in Table 11, it was recognized that the coating of the essence made according to the embodiment 4 had a higher moisturizing capacity than the coating of the essence made according to the comparative example 1.

Referring to the experimental examples 1 to 10 and the tables 1 to 11, the cosmetic made from the composition of the present invention may become thick without using a conventional thickener, so stable and less skin stimulations can be obtained, and it does not flow down while providing a high moisturizing effect, so it is easy to carry and use. In addition, it feels sticky less and a tightening feeling is small when in use, so the use feels good.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

### LEGEND OF REFERENCE NUMBERS

1: Embodiment 1
2: Embodiment 2
3: Embodiment 3
4: Embodiment 4
5: Embodiment 5
6: Comparative example 1
7: Comparative example 2

What is claimed is:

1. A transparent sol and gel composition containing high concentrations of vitamin C and vitamin C derivative, comprising:
0.2˜20% by weight of vitamin C or vitamin C derivative;
20˜80% by weight of polyhydric alcohol;
0.05˜3% by weight of acidic mucopolysaccharide; and
15˜60% by weight of a hydrophilic solvent.

2. The composition of claim 1, wherein the polyhydric alcohol contains at least one of glycol, 1,3-butylene glycol, propylene glycol, sorbitol and glycerin.

3. The composition of claim 1, wherein the acidic mucopolysaccharide contains at least one of hyaluronic acid natrium, chondroitin sulfuric acid, and heparin.

4. The composition of claim 1, wherein the hydrophilic solvent is a viscosity adjusting solvent and contains at least one of distilled water and ethyl alcohol.

5. The composition of claim 1, wherein the vitamin C derivative consist of at least one of sodium ascorbylphosphate (SAP), magnesium ascorbylphosphate (MAP), calcium ascorbylphosphate, ascorbic acid polpeptide, ascorbyl dipalmitate, ascorbyl palmitate, ascorbyl glucoside, ethyl ascorbyl ether, ascorbyl stearate and ascorbyl ethylsilanolpectinate.

6. The composition of claim 4, wherein the hydrophilic solvent when it is used mixed with distilled water and ethyl alcohol comprises:
15˜40% by weight of distilled water; and
0˜20% by weight of ethyl alcohol.

7. The composition of claim 1, wherein the composition has a formulation, for example, essence, a mask pack, a hydrogel mask, etc.
